(19) United States
(12) Patent Application Publication
(10) Pub. No.: US 2021/0298645 A1
(43) Pub. Date: Sep. 30, 2021

(54) A REHABILITATION DEVICE AND A METHOD OF MONITORING HAND MOVEMENT

(71) Applicant: KONINKLIJKE PHILIPS N.V., EINDHOVEN (NL)

(72) Inventors: Jin WANG, Shanghai (CN); Sheng JIN, Shanghai (CN); Wei LI, Shanghai (CN); Xiang WANG, Shanghai (CN); Yu ZHANG, Shanghai (CN)

(21) Appl. No.: 17/260,262

(22) PCT Filed: Jul. 8, 2019
(86) PCT No.: PCT/EP2019/068189

§ 371 Date: Jan. 14, 2021

(30) Foreign Application Priority Data
Jul. 18, 2018 (CN) PCT/CN2018/096086
Sep. 20, 2018 (EP) 18195743.2

(51) Int. Cl. A61B 5/11; A63B 23/16; A61B 5/00; A63B 71/06; A63B 24/00

(52) U.S. Cl. CPC A61B 5/1124; A63B 23/16; A61B 5/1128; A63B 2220/806; A63B 71/0622; A63B 24/0006; A61B 2505/09; A61B 5/702

(57) ABSTRACT

A rehabilitation device is for monitoring hand movement of a user in which one or both forearms are supported on a forearm support region of a support, and an upward facing camera is mounted with a field of view forwards beyond the front of the forearm support to capture an image of the hand or hands, which extend in front of the forearm support region. This avoids the need for the user to wear a sensor glove but also enables a compact system without needing an overhead camera or a large table arrangement.

### FIELD OF THE INVENTION

This invention relates to a rehabilitation device, and in particular to a hand rehabilitation device which monitors movement of a user's hand.

### BACKGROUND OF THE INVENTION

Movement therapy is used to rehabilitate patients who have diminished motor control; for example, movement therapy may be suitable for a stroke patient who has reduced control of their hand. Rehabilitation devices are used to facilitate movement therapy. These devices may guide, monitor and measure how patients conduct exercises, give feedback on exercise quality and motivate patients to comply with a training plan.

Some rehabilitation devices use video cameras to track a user's hand movements, whilst other rehabilitation devices use active sensors to detect hand movements. Active sensor rehabilitation devices are, in general, more expensive than video camera rehabilitation devices. Furthermore, an active sensor system typically requires a glove to be worn by the user, which may be uncomfortable as a result of the injury being treated.

For a camera based system, a tracking system may be used which includes a video camera for capturing hand movement in the RGB color field and a depth sensor for obtaining depth information. The video camera records an image of the patient's hand, which is processed to localize the hand in the image and to segment the hand from the background of the image, in order to track the position of the patient's hand. Segmentation may be performed based on color, since skin color may be easily distinguished from the background of the image. The patient may wear colored markers to improve the accuracy of detection. Other 3D camera designs are also known.

A problem with camera based systems is that they take up a lot of space and are not easily portable. For example, a camera is typically mounted on a frame over the top of a table which has a support for the user's forearms.

There is therefore a need for an improved hand rehabilitation device which is more compact and portable.

### SUMMARY OF THE INVENTION

The invention is defined by the claims.

According to examples in accordance with an aspect of the invention, there is provided a rehabilitation device for monitoring hand movement of a user, comprising:

a frame having a display mounting for attachment of a display;

a support mounted to the frame, wherein the support comprises a base and a top, the top having a forearm support region; and

a camera mounted on the base facing the top, such that a field of view of the camera comprises a region which extends in front of the forearm support region.

This device provides a compact way to image the hands of a user, by arranging the hand (or hands) to overhang the end of a forearm support region. The hand projects to a region in front of the forearm support region, and hence into the field of view of the camera. The device provides imaging of the hand or hands from below. It avoids the need for the user to wear a sensor glove but also enables a compact system without needing an overhead camera or a large table arrangement.

The camera is at a fixed position relative to the hand or hands of the user and it is in relatively close proximity. As a result, the camera does not need a large field of view, and its field of view may extend only slightly beyond the volume occupied by the hands of the user.

The support may further comprises a hand support region. This may be used to support the wrist and part of the palm near the wrist. This may provide support for a user who does not have sufficient strength to support their hand when only a forearm support is provided.

At least part of the hand support region is preferably made of a transparent material. This enables an image to be taken through the support.

As mentioned above, the hand support region may be for supporting the wrist and part of the palm of the hand. The device may further comprise a second hand support region for supporting the remainder of the palm of the hand, wherein the second hand support region is removably mounted.

This means the device can be adapted to the needs of different users, having different requirements for the support of their hand or hands. More freedom to perform exercise movements is available without the second hand support region, since more vertical space is available to do different types of movements. The second hand support region may fully support the hand, by providing support for the fingers as well.

The frame may comprise an upright member, wherein the support is mounted to the upright member with adjustable height.

This enables the user to position the support in the most comfortable way. The camera is attached to the support so that this adjustment does not need any recalibration of the camera. Of course, the user may additionally or alternatively adjust the height of a chair they are sitting on.

The support may be mounted to the upright member with an adjustable elevation angle.

This angle may be used to set different types of rehabilitation exercise and difficulty. Again, because the camera moves with the support, this requires no recalibration of the camera.

The frame may comprise an upright member, wherein the display mounting has an adjustable height along the upright member. Thus, the display may have an adjustable height, for best comfort of the user.

The frame may comprise a wheeled trolley.

The device is then a portable unit which can be wheeled to any location. The user simply brings a chair to sit in front of the device and perform the rehabilitation procedure.

The forearm support region may comprise two forearm support channels which are spaced apart and taper inwardly in a direction towards the front surface.

This provides a comfortable rest position for the arms and hands of the user.

The base and the top are for example spaced by between 10 cm and 30 cm. This spacing is sufficient for the camera to have a field of view of the volume occupied by the hands but also enables a compact overall device.

The top is for example removable from the base. The top may be removed for cleaning, but also there may be different tops to suit people with different arm sizes or even different angular positions of the arms which are most comfortable, or different arm positions for different exercises.

The base may comprise a tab which projects forwards from a front of the base, and the camera is mounted on the tab. The camera is thus mounted in front of the forearm support region facing upwardly to the volume where the overhanging (supported by the hand support region or unsupported) hands will be located. This provides a compact arrangement.

Alternatively, the base may be larger, and the camera is mounted at a central area of the base.

The camera may comprise a 3D camera. There are commercially available suitable depth sensing cameras, such as the Leap Motion (trade mark) camera.

The device may comprise a display mounted to the display mounting. The device then includes the display. The device thus includes all the units required for performing hand rehabilitation, other than a chair on which the user can sit.

The device preferably further comprises a controller, which is adapted to:

generate a display output comprising instructions for a user to perform particular hand rehabilitation exercises;

analyze movement of a hand or both hands of the user; and

generate an output which indicates a success level in performing the hand rehabilitation exercises.

By assessing the performance of the user in performing the rehabilitation exercises, the motivation of the user can be improved.

The invention also provides a method of monitoring hand movement of a user of a rehabilitation system, comprising:

capturing images of one or both hands of the user when the users arms are resting on a support which comprises a base and a top, the top having a forearm support region, wherein the user's hand or hands extend in front of the forearm support region,

wherein the images are captured using a camera which is mounted on the base facing the top, such that a field of view of the camera comprises a region which extends in front of the forearm support region in which the user's hands are present.

Capturing images may comprise capturing 3D images.

The invention also provides a a rehabilitation method, comprising:

generating a display output comprising instructions for a user to perform particular hand rehabilitation exercises;

monitoring hand movement using the method described above;

analyzing the hand movement; and

generating an output which indicates a success level in performing the hand rehabilitation exercises.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a rehabilitation device according to an example;

FIG. 2 shows the orientation of the user when using the device of FIG. 1;

FIG. 3 shows a first alternative design of the support used in the system of FIG. 1; and

FIG. 4 shows a second alternative design of the support used in the system of FIG. 1.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a rehabilitation device for monitoring hand movement of a user in which one or both forearms are supported on a forearm support region of a support, and an upward facing camera is mounted with a field of view forwards beyond the front of the forearm support to capture an image of the hand or hands. The hand or hands extend in front of the forearm support region. This avoids the need for the user to wear a sensor glove but also enables a compact system without needing an overhead camera or a large table arrangement.

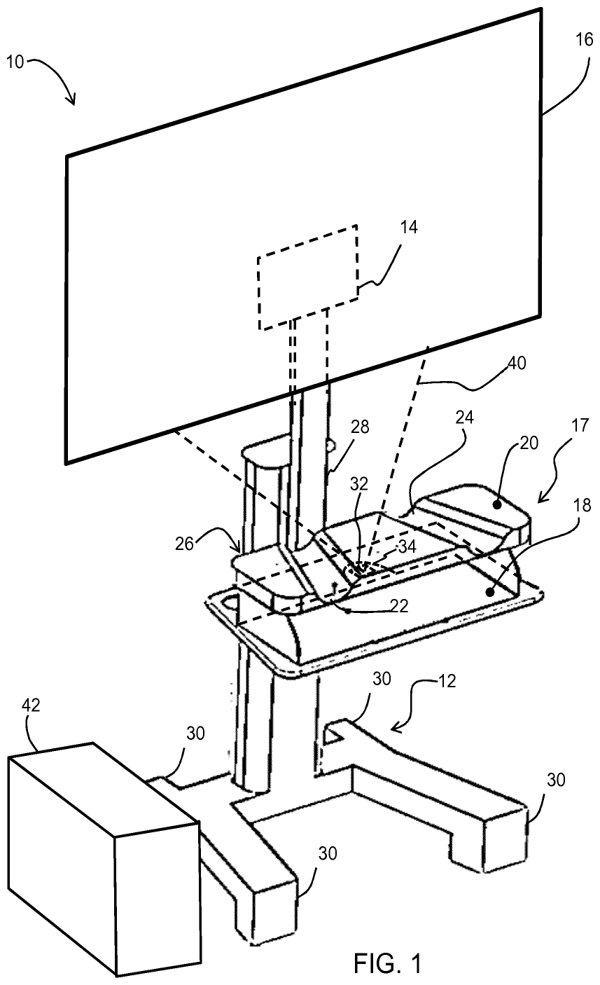

FIG. 1 shows a rehabilitation device 10 for monitoring hand movement of a user. It comprises a frame 12 having a display mounting 14 for attachment of a display 16. The display 16 is positioned in front of and above a support 17 also mounted to the frame 12. The support 17 comprises a base 18 and a top 20. The support is mounted on a shelf.

FIG. 1 shows a first possible design of the support 17. In this design, the top 20 of the support comprises two forearm support channels 22, 24 which end at a front surface 26 of the top.

The two forearm support channels 22, 24 are spaced apart and taper inwardly in a direction towards the front surface 26. This provides a comfortable rest position for the arms and hands of the user.

The base and the top are for example spaced by between 10cm and 30cm. This spacing is sufficient for the camera to have a field of view of the volume occupied by the hands but also enables a compact overall device.

The frame 12 has an upright member 28, and the support 17 is mounted to the upright member with adjustable height (i.e. the shelf may have adjustable height). Similarly, the display 16 may optionally also have an adjustable height on the frame 12.

The support 17 may be mounted to the upright member 28 with an adjustable elevation angle (i.e. the shelf may have adjustable angle). This angle may be used to set different types of rehabilitation exercise and difficulty.

In the example shown, the frame 12 is a wheeled trolley having four feet 30 which have rollers at their underside (not shown). The device is then a portable unit which can be wheeled to any location. The user simply brings a chair to sit in front of the device and perform the rehabilitation procedure.

In the example of FIG. 1, a camera 32 is mounted on a tab 34 which is part of the base 18. The camera 32 faces the top, such that a field of view 40 of the camera comprises a region which extends forwards beyond the front surface 26. This is where the user's hand or hands are located when using the device.

A transparent material cover may be provided over the camera for example to prevent water ingress. The transparent part may only cover the camera, for other parts the materials can be opaque.

This device provides a compact way to image the hands of a user, by arranging the hands to overhang the end of the support 17. The hands project into the field of view 40 of the camera.

The camera is at a fixed position relative to the hands of the user and it is in relatively close proximity. As a result, the camera does not need a large field of view 40, and its field of view may extend only slightly beyond the volume occupied by the hands of the user.

Preferably, an image of the whole hand is captured by the camera in order to be able to conduct hand gesture recognition.

The camera is attached to the support so that adjustment of the forearm support does not need any recalibration of the camera.

The device comprises a controller 42 which is used to analyze the capture images and display information relating to the rehabilitation. For example, the controller generates a display output comprising instructions for a user to perform particular hand rehabilitation exercises. The movement of a hand or both hands of the user is then analyzed. An output is then provided which indicates a success level in performing the hand rehabilitation exercises.

As is well known, the exercises may be modified in dependence on the performance of the user. The exercise may involves a game play situation to make the experience more entertaining. This invention is not concerned with the rehabilitation procedure itself so further details will not be provided.

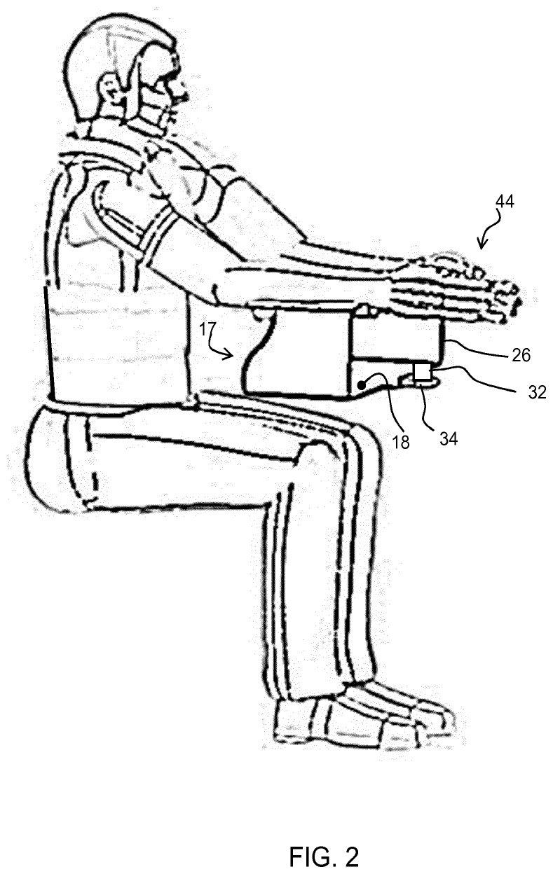

FIG. 2 shows the position of the user when using the device. As shown, the user's hands 44 overlap, i.e. project beyond, the end face 26 of the support 17. FIG. 2 also shows the camera 32 more clearly, and the tab 34 which projects forwards from the front of the base 18.

The camera may comprise a 3D camera. There are commercially available suitable depth sensing cameras, such as the Leap Motion (trade mark) camera. The 3D camera with uses an array of infrared LEDs and has a filtered camera lens. Image processing enables the camera to segment the hand out of the background image and recognize hand gestures.

FIGS. 1 and 2 show a first example of the support 17, for supporting both forearms and in which the whole of the support 17 may be considered to be a forearm support region.

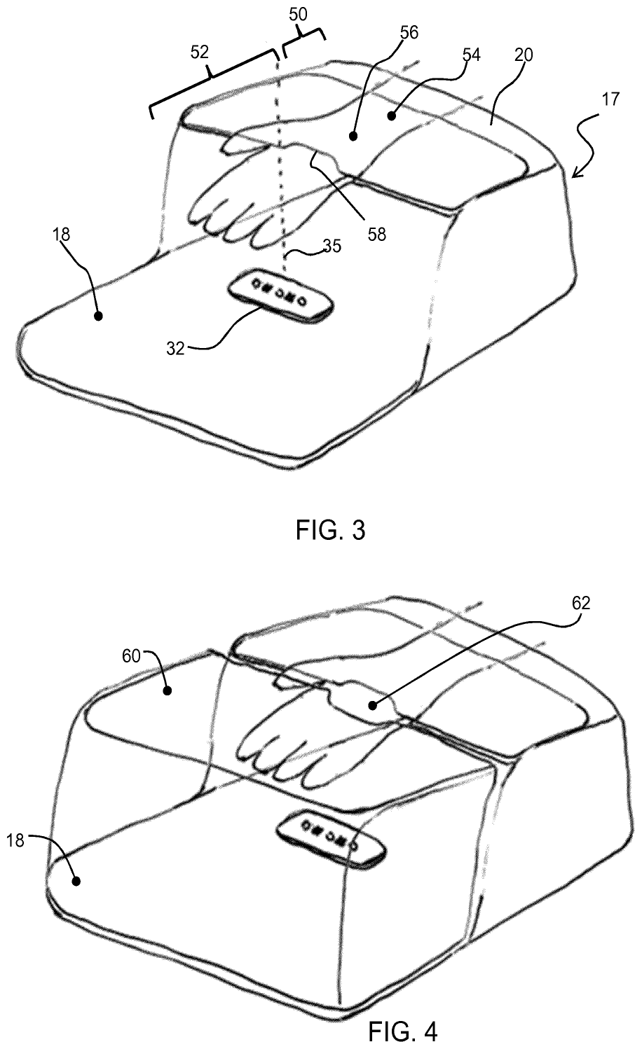

FIG. 3 shows a second example, for only one hand. Often, a patient is paralyzed on one side of their body so that only one hand needs to carry out the exercise. Of course, a patient may simply use one channel of the support of FIG. 1, but a single hand support is also possible as shown in FIG. 3.

Furthermore, in this example, the support extends further forward so that there is a forearm support region 50 and a hand support region 52. The hand support region 52 comprises a rigid and transparent material so that it provides support for the hand and also enables images to be captured through the hand support region 52. The hand support region 52 supports the wrist 54 and part of the palm 56 near the wrist side.

As explained above, the top 20 (and hence also the hand support region which is part of the top) is elevated by a certain height above the base 18. The camera 32 again faces upwardly, for example directed to the center of the palm 56.

The whole hand including part of the wrist is in the field of view of the camera to make the hand gesture recognition accurate. The support to the lower part of the palm and part of the wrist enables hand rehabilitation with weak muscle strength.

The camera includes an illumination source for the imaging function, such as infrared LEDs, which project illumination light upwardly as represented by the line 35. The hand support region does not extend so far forward that it extends directly over the infrared LEDs of the camera. In this way, it is prevented that the hand support region 52 provides direct reflections of the illumination light to the camera and hence affect the hand gesture recognition.

In the example shown, the hand support region has a notch 58 directly above the camera.

The hand support region 52 may be removable so that support is then only provided as a forearm support region 50.

FIG. 4 shows a modification in which a second hand support region 60 is provided. This is an accessory which can also be mounted on the base 18 with the same height as the first hand support region 52. It supports the remainder of the palm and also the fingers. Thus, the second hand support region 60 is removably mounted, and optionally also the first hand support region is removably mounted.

To avoid direct reflection as explained above, an opening 62 is provided over the illumination LEDs of the camera. Thus, the area which would cause unwanted direct refection is left open. This combined full support solution is appropriate for weak patients who need a full support of the hand to perform the hand rehabilitation. The full support may affect the ability to perform certain exercises since the second hand support region will block some finger movement, however, other unaffected exercises can be done using the full support for weaker patients.

The top 20 is for example removable from the base 18. The top may be removed for cleaning, but also there may be different tops to suit people with different arm sizes or even different angular positions of the arms which are most comfortable, or different arm or hand positions for different exercises.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A rehabilitation device for monitoring hand movement of a user, comprising:
a frame having a display mounting for attachment of a display;
a support mounted to the frame, wherein the support comprises a base and a top, the top having a forearm support region; and
a camera mounted on the base facing the top, such that a field of view of the camera comprises a region which extends in front of the forearm support region.

2. A device as claimed in claim 1, wherein the support further comprises a hand support region.

3. A device as claimed in claim 2, wherein at least part of the hand support region is made of a transparent material.

4. A device as claimed in claim 2 wherein the hand support region is for supporting the wrist and part of the palm of the hand, wherein the device further comprises a second hand support region for supporting the remainder of the palm of the hand, wherein the second hand support region is removably mounted.

5. A device as claimed in claim 1, wherein the frame comprises an upright member, wherein:
the support is mounted to the upright member with adjustable height and/or
the display mounting has an adjustable height along the upright member; and/or
the support is mounted to the upright member with an adjustable elevation angle.

6. A device as claimed in claim 1, wherein the forearm support region comprises two forearm support channels which are spaced apart and taper inwardly in a direction towards the front surface.

7. A device as claimed in claim 1, wherein the base and the top are spaced by between 10 cm and 30 cm.

8. A device as claimed in claim 1, wherein the top is removable from the base.

9. A device as claimed in claim 1, wherein the base comprises a tab which projects forwards from a front of the base, and the camera is mounted on the tab.

10. A device as claimed in claim 1, wherein the camera comprises a 3D camera.

11. A device as claimed in claim 1, comprising a display mounted to the display mounting.

12. A device as claimed in claim 1, further comprising a controller, which is adapted to:
generate a display output comprising instructions for a user to perform particular hand rehabilitation exercises;
analyze movement of a hand or both hands of the user; and
generate an output which indicates a success level in performing the hand rehabilitation exercises.

13. A method of monitoring hand movement of a user of a rehabilitation system, comprising:
capturing images of one or both hands of the user when the users arms are resting on a support which comprises a base and a top, the top having a forearm support region, wherein the user's hand or hands extend in front of the forearm support region,
wherein the images are captured using a camera which is mounted on the base facing the top, such that a field of view of the camera comprises a region which extends in front of the forearm support region in which the user's hands are present.

14. A method as claimed in claim 13, wherein capturing images comprises capturing 3D images.

15. A rehabilitation method, comprising:
generating a display output comprising instructions for a user to perform particular hand rehabilitation exercises;
monitoring hand movement using the method of claim 13,
analyzing the hand movement; and
generating an output which indicates a success level in performing the hand rehabilitation exercises.
